(19) United States
(12) Patent Application Publication
(10) Pub. No.: US 2012/0123425 A1
(43) Pub. Date: May 17, 2012

(54) Joint Manipulator Device

(76) Inventor: Logan A. Worley, Sterling Heights, MI (US)

(21) Appl. No.: 13/294,157

(22) Filed: Nov. 10, 2011

Related U.S. Application Data
(60) Provisional application No. 61/412,417, filed on Nov. 11, 2010.

(51) Int. Cl. A61B 17/56

(52) U.S. Cl. 606/90

(57) ABSTRACT

The present invention relates to a joint manipulator for rehabilitation of an appendage that has undergone joint replacement surgery or can otherwise benefit from repetitious movement. In one exemplary embodiment, the present invention provides a joint manipulator device for engagement and manipulation of an appendage of an individual. The joint manipulator includes an appendage coupler defining an appendage aperture for receiving a toe of an individual. The joint manipulator further includes a handle disposed with respect to the appendage coupler for causing movement of the appendage coupler. The joint manipulator further includes one or more extension rods connecting the handle to the appendage coupler, wherein upon movement of the handle the appendage coupler moves the toe with respect to an associated foot.

### CROSS REFERENCE TO RELATED APPLICATION

This application claims priority to U.S. Provisional Application No. 61/412,417 which was filed on Nov. 11, 2011, the contents of which are hereby incorporated by reference in its entirety for all purposes.

### FIELD OF THE INVENTION

The present invention relates to a joint manipulator for rehabilitation of an appendage that has undergone joint replacement surgery or can otherwise benefit from repetitious movement.

### BACKGROUND/DESCRIPTION OF PRIOR ART

Deteriorated and damaged joints can cause mild to immense pain to an individual. Under certain circumstances, joint replacement is the only recourse to alleviate pain and regain function of the joint. However, replacement of joints through surgery results in the formation of scar tissue about the new joint. To this end, it is important that after a specified healing time has elapsed, the joint is manipulated, flexed, stretched or otherwise moved about to minimize the amount of scar tissue formed about the joint. Similarly, individuals having arthritis also benefit from manipulation, flexing, stretching or other movement of an arthritic joint. However, as joint deterioration is often an age related occurrence, many individuals undergoing joint replacement and arthritis have mobility limitations.

In one particular instance, it is becoming more common for individuals to have joint replacements of lower appendages such as ankle and toe joints. Similarly, arthritis also effects lower appendages. With individuals having limited mobility, it is difficult to manipulate the replaced or arthritic joint. Also, care must be given during manipulation as the surrounding area to the replaced joint may be sensitive.

Accordingly, there is a need for improved methods and devices for manipulation of joints of lower extremities that accounts for mobility limitations and sensitivity of the surrounding area of the joint.

### SUMMARY OF THE INVENTION

The present invention relates to methods and devices for the manipulation of artificial joints after surgery and other joints that may benefit from repetitious movement. The advancements of the present invention are predicated, at least in part, upon the use of a device that includes a coupling device for an appendage disposed at a first end of a rod and a handle disposed at a second end of the rod for grasping and manipulation of the coupling device and hence the appendage.

In greater detail, in one aspect, the present invention provides a joint manipulator device for engagement and manipulation of an appendage of an individual. The joint manipulator includes an appendage coupler defining an appendage aperture for receiving a toe of an individual. The joint manipulator further includes a handle disposed with respect to the appendage coupler for causing movement of the appendage coupler. The joint manipulator further includes one or more extension rods connecting the handle to the appendage coupler, wherein upon movement of the handle the appendage coupler moves the toe with respect to an associated foot.

The above-described and other features and advantages of the present invention will be appreciated and understood by those skilled in the art from the following detailed description, drawings, and appended claims.

### BRIEF DESCRIPTION OF THE DRAWINGS

Other objects, features, advantages and details of the present invention appear, by way of example only, in the following detailed description of preferred embodiments of the invention, the detailed description referring to the drawings in which:

FIG. 1 illustrates an elevational view of a joint manipulator according to an exemplary embodiment of the present invention;

FIG. 2 illustrates the joint manipulator shown in FIG. 1 being used by an individual according to an exemplary method of use;

FIG. 3 illustrates a perspective view of an appendage coupler according to an exemplary embodiment of the present invention;

FIG. 4 illustrates an elevational view of the appendage coupler shown in FIG. 3;

FIG. 5 illustrates another elevational view of the appendage coupler shown in FIG. 3;

FIG. 6 illustrates a cross-sectional view of the appendage coupler shown in FIG. 4;

FIG. 7 illustrates an enlarged view of an appendage coupler attachment feature shown in FIG. 6;

FIG. 8 illustrates a perspective view of a handle according to an exemplary embodiment of the present invention;

FIG. 9 illustrates an elevational view of the handle shown in FIG. 8;

FIG. 10 illustrates another elevational view of the handle shown in FIG. 8;

FIG. 11 illustrates another elevational view of the handle shown in FIG. 8; and

FIG. 12 illustrates a cross-sectional view of the handle shown in FIG. 9.

### DETAILED DESCRIPTION OF THE PREFERRED EMBODIMENT

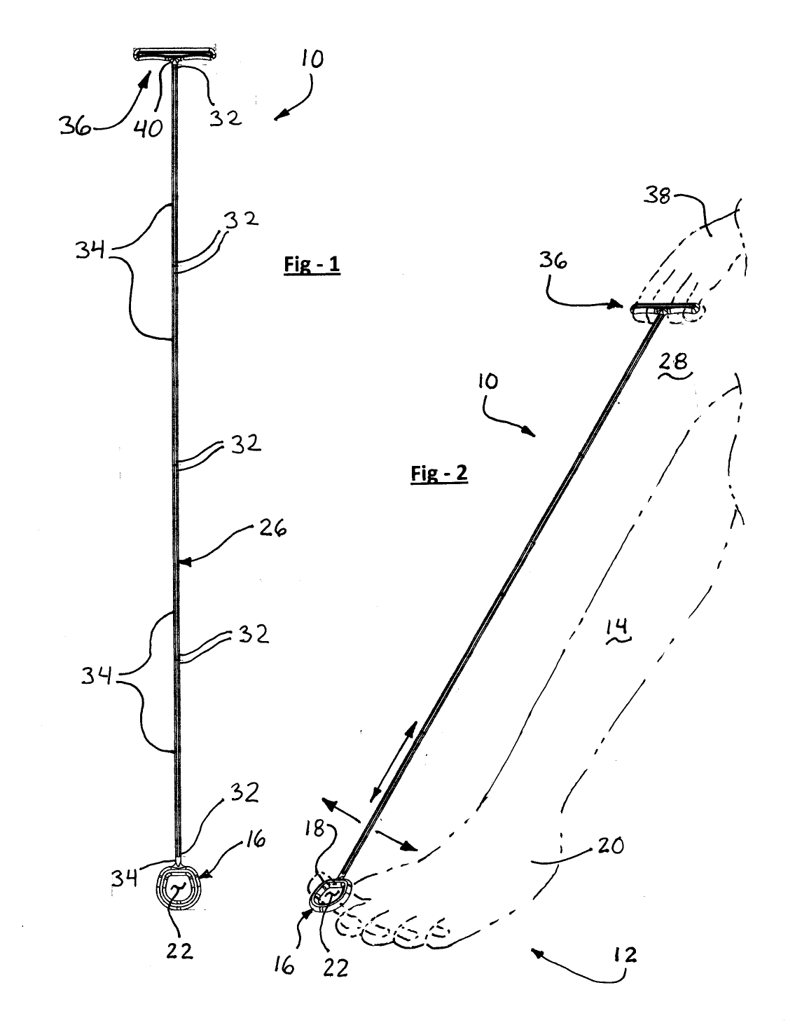

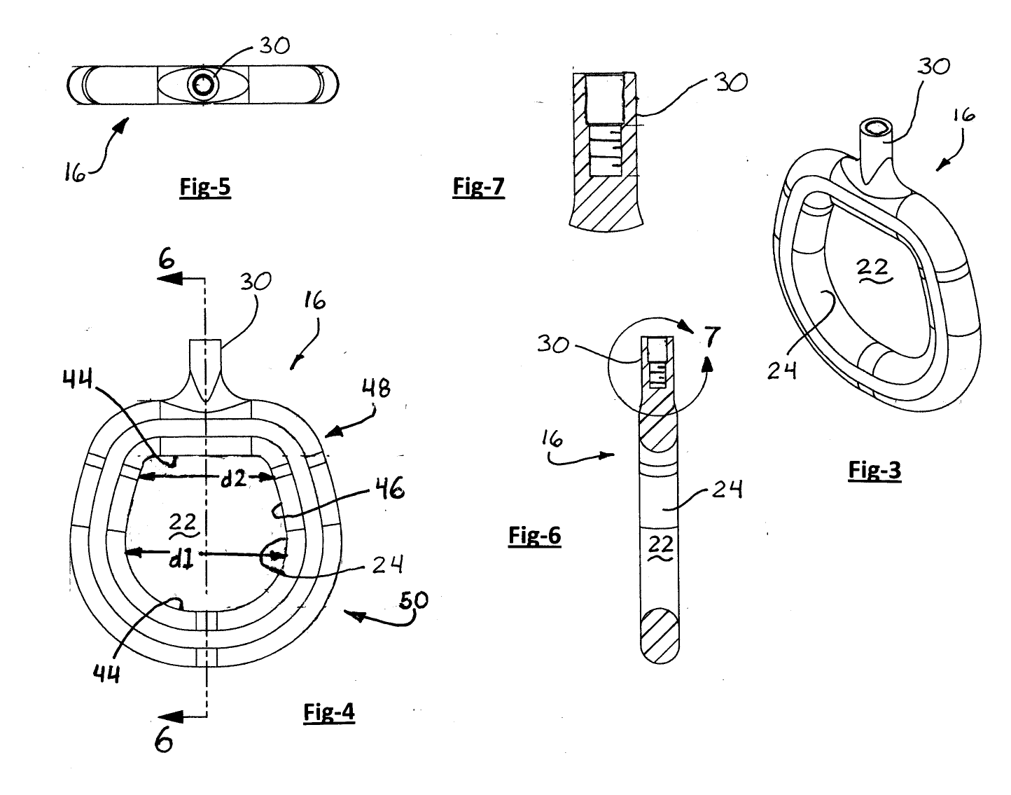

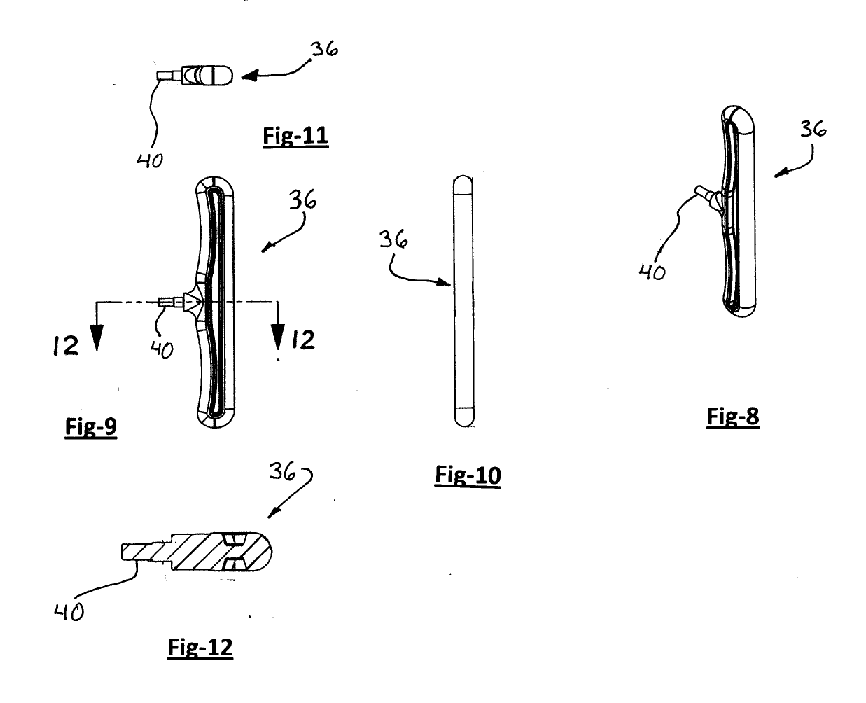

Referring to FIGS. 1 and 2, the present invention provides a joint manipulator device 10 configured for engagement with an appendage 12 of an individual 14. In the embodiment shown, the joint manipulator includes an appendage coupler 16 configured for engagement with a toe 18 of the individual 14. However, in another exemplary embodiment the appendage coupler is configured for engagement with another appendage such as a foot 20 or otherwise. Referring again to the embodiment shown in FIGS. 1 and 2 and shown in FIGS. 3 through 7, the appendage coupler 16 includes an appendage aperture 22 that is suitable in size and shape for receiving the toe 18. Advantageously, the appendage aperture 22 is defined by internal walls 24 that are capable of engaging a top portion, bottom portion and side portions of the toe 18 for manipulation thereof, and hence a corresponding joint, in multiple directions. In one exemplary embodiment, the internal walls 24 include a generally flat top wall 42, an arcuate bottom wall 44, and side walls 46 that converge towards the top wall 42. This convergence forms a first diameter d1 at a top portion 48 of the appendage aperture 22 and a second diameter d2 at a bottom portion 50 of the appendage aperture 22, wherein the first diameter d1 is greater than the second diameter d2.

In one exemplary embodiment, the internal walls are of formed of or include a layer of flexible and resilient material, such as foam, sponge, cloth or other soft material, for providing a cushion between the device 10 and toe. It a contemplated that the appendage coupler 16 is capable of providing horizontal movement, lateral movement and diagonal movement of the corresponding joint. Further, due to the surrounding nature of the appendage coupler 16, the joint may further be manipulated in a circular or oval pattern to obtain additional desired movement of the joint. Other manipulation is possible.

The appendage coupler 16 is attached to an extension, such as rod 26, to provide the ability to manipulate the toe 18 from a distal region, such as proximate a knee region 28, or otherwise, that is convenient for a user of the joint manipulator device 10. In one exemplary embodiment, the appendage coupler 16 includes an attachment feature 30 for releasable attachment to the rod 26. Advantageously, this provides the ability to disassemble the joint manipulator device 10 for convenient carrying and stowage. In one configuration, the releasable attachment of the attachment feature 30 comprises a threaded fastener formed between the appendage coupler 16 and a corresponding attachment feature 32 of the rod 26. Other releasable attachment configurations are possible.

In one exemplary embodiment, the rod 26 comprises a plurality of individual segments 34 each including attachment features 28 configured for engagement to one another, the appendage coupler 16 or a handle 36, as described below. The number of segments 34 can be based upon a desired length of the entire rod 26 or joint manipulator 10. In one particular embodiment, the rod includes a cross-sectional shape to facilitate individuals, particularly weak or arthritic individuals, in disconnecting the individual segments from each other or the rod 26 from the appendage coupler 16 and/or handle 36, e.g. through twisting or otherwise. In one configuration, the rod includes a cross-sectional shape that is triangular, square, pentagonal, hexagonal (as shown in FIGS. 1 and 2), heptagonal, octagonal or other contoured shape.

In one exemplarity embodiment, it is contemplated that the entire length of the rod 26, or joint manipulator 10, is at least 12 inches, 18 inches, 24 inches or more. In another exemplarity embodiment, it is contemplated that the entire length of the rod 26, or joint manipulator 10, is between about 18 inches to about 30 inches. Advantageously, this provides the ability of an individual, with limited mobility, to manipulate the joint without undue stress on other parts of the body including back, neck, shoulder, arm or otherwise. As should be appreciated, a user may use a desired amount of segments 34 to obtain a desired length of the rod 26 and hence joint manipulator 10.

As previously mentioned, it is contemplated that in one exemplary embodiment the joint manipulator 10 includes handle 36 for manipulation of the appendage coupler 16. Referring to FIGS. 1 and 2 and FIGS. 8 through 12, an exemplary handle 36 is shown. In this configuration, the handle 36 is particularly configured for grasping by a hand 38 of the individual 14. In this configuration, the handle is T-shaped and includes an attachment features 40 for releasable attachment with rod 22, or segment 34 thereof. In one particular configuration, the releasable engagement is achieved through a threaded engagement between the handle 36 and rod 26 or segment 34. Advantageously, this further provides the ability to stow and carry the joint manipulator 10.

The joint manipulator device 10 may be formed of any suitable material for manipulation of a joint. In one exemplary embodiment, the components of the joint manipulator device 10 are semi-rigid or rigid to minimize flex of the device during use. In another exemplary embodiment, the components of the joint manipulator device 10, in particular the extension rods, appendage coupler or both, are flexible to reduce jarring movement during joint manipulation. Examples of suitable materials that may be used for one or more components of the joint manipulator device 10 include plastic, metal, ceramic or otherwise. It is contemplated that the components of the joint manipulator device 10 may be formed of similar or dissimilar materials.

In one exemplary use of the joint manipulator device 10, the appendage coupler 16, rod 26 and handle 36 is removed from a bag or other carrying and stowage device, not shown, and assembled as shown in FIGS. 1 and 2. The individual 14 positions the appendage coupler proximate to toe 18 and moves the toe or otherwise causing the toe to be positioned within the appendage aperture 22. The individual utilizes the handle to engage one or more sides of the toe 18 to manipulate the toe 18 in a desired fashion to cause suitable flexing of the joint of the toe 18.

It is contemplated that the joint manipulator device 10 may include multiple appendage couplers 16 having different sizes and shapes for engagement with different appendages and may be selected based upon the application. Similarly, the user may select a number of segments 34 to obtain a desired length of the manipulator device 10. Still further, the joint manipulator device 10 may include multiple handles 36 to allow a user to select a handle best suited for him or her.

While the invention has been described with reference to a preferred embodiment it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A joint manipulator device for engagement and manipulation of an appendage of an individual, comprising:
an appendage coupler defining an appendage aperture for receiving a toe of an individual;
a handle disposed with respect to the appendage coupler for causing movement of the appendage coupler; and
one or more extension rods connecting the handle to the appendage coupler, wherein upon movement of the handle the appendage coupler is configured for movement of the toe with respect to an associated foot.

2. The device of claim 1, wherein the appendage coupler includes a releasable attachment feature for attachment to the one or more extension rods.

3. The device of claim 1, wherein the appendage aperture includes a shape and size that generally corresponds to the shape of a toe.

4. The device of claim 3, wherein the appendage aperture has a first diameter disposed proximate a bottom portion of the appendage aperture that is greater than a second diameter disposed proximate a top portion of the appendage aperture.

5. The device of claim 3, wherein the appendage aperture has a top wall that is generally flat and a bottom wall that is arcuate.

6. The device of claim 3, wherein the appendage coupler includes internal walls forming and substantially surrounding the appendage aperture.

7. The device of claim 1, wherein one or more extension rods comprise a plurality of extension rods, each of the plurality of extension rods including a releasable attachment feature for attachment to one another and for attachment to the appendage coupler and the handle.

8. The device of claim 7, wherein the attachments feature of the plurality of extension rods comprises threaded fasteners.

9. The device of claim 1, wherein the handle comprises a t-shaped member including an attachment feature for releasable attachment to the one or more extension rods.

10. The device of claim 1, wherein the entire length of the one or more extension rods is at least 12 inches.

11. The device of claim 1, wherein the entire length of the joint manipulator device is between about 18 inches to about 30 inches.

12. The device of claim 1, wherein the one or more extension rods are flexible and resilient.

13. The device of claim 11, wherein the one or more extension rods are formed of plastic.

14. The device of claim 1, wherein the appendage coupler includes one or more internal walls formed of a flexible and resilient material.

15. The device of claim 1, wherein the material forming the internal walls of the appendage coupler comprises foam.

16. The device of claim 1, further comprising a carrying device for storage and carrying of the components of the joint manipulator device.

17. The device of claim 1, wherein the one or more extension rods include a non-circular contoured cross-sectional shape.

18. A joint manipulator device for engagement and manipulation of an appendage of an individual, comprising:
an appendage coupler defining an appendage aperture suitable in size and shape for receiving a toe of an individual;
a handle disposed with respect to the appendage coupler for causing movement of the appendage coupler; and
a plurality of extension rods connecting the handle to the appendage coupler, the plurality of extension rods having a non-circular cross-sectional contour and including releasable attachment features for attachment to one another and for attachment to the appendage coupler and the handle, wherein upon movement of the handle the appendage coupler is configured for movement of the toe with respect to an associated foot.

19. The device of claim 18, wherein the appendage coupler includes internal walls forming and substantially surrounding the appendage aperture, the internal walls including a top wall that is generally flat and a bottom wall that is arcuate and two side walls converging towards the top portion, wherein the appendage aperture has a first diameter disposed proximate a bottom portion of the appendage aperture that is greater than a second diameter disposed proximate a top portion of the appendage aperture.

20. The device of claim 19, wherein the handle comprises a t-shaped member including an attachment feature for releasable attachment to the one or more extension rods and wherein the entire length of the device is between about 18 inches to about 30 inches.
